(19) United States
(12) Patent Application Publication
(10) Pub. No.: US 2025/0113808 A1
(43) Pub. Date: Apr. 10, 2025

(54) BRAIN-METASTATIC LUNG CANCER CELL LINE AND USE OF NOGGIN AS MARKER FOR BRAIN METASTASIS OF LUNG CANCER

(71) Applicants: THE CATHOLIC UNIVERSITY OF KOREA INDUSTRY-ACADEMIC COOPERATION FOUNDATION, Seoul (KR); EWHA UNIVERSITY - INDUSTRY COLLABORATION FOUNDATION, Seoul (KR)

(72) Inventors: Seung Ho YANG, Seoul (KR); Young-Ho AHN, Seoul (KR); Jung Eun LEE, Gyeonggi-do (KR)

(21) Appl. No.: 18/906,789

(22) Filed: Oct. 4, 2024

(30) Foreign Application Priority Data
Oct. 6, 2023 (KR) 10-2023-0132976

(51) Int. Cl. A01K 67/0271; A61K 38/17; A61K 48/00; C12Q 1/6886; G01N 33/574

(52) U.S. Cl. CPC A01K 67/0271; A61K 38/1709; C12Q 1/6886; G01N 33/57423; A01K 2207/12; A01K 2267/0331; A61K 48/00; C12Q 2600/158

(57) ABSTRACT

The present invention relates to a brain-metastatic lung cancer cell line and a use of Noggin as a marker for brain metastasis of lung cancer. In the present invention, a brain metastatic lung cancer cell line with optimized characteristics for brain metastasis was established by repeatedly transplanting the lung cancer cell line through the carotid artery. In addition, in the brain metastatic lung cancer cell line, the expressions of Noggin protein and its mRNA are reduced, confirming that the motility and invasiveness of cells are reduced and adhesion increased. Therefore, the Noggin protein or its gene is expected to be useful as a biomarker for diagnosis of brain metastasis of lung cancer and its target therapy.

### CROSS REFERENCE TO RELATED APPLICATIONS

This application claims priority to and the benefit of Korean Patent Application No. 10-2023-0132976, filed on Oct. 6, 2023, the disclosure of which is incorporated herein by reference in its entirety.

### REFERENCE TO A “SEQUENCE LISTING,” SUBMITTED AS AN XML FILE

The Sequence Listing written in the xml file titled: “206132-0181-00US_SequenceListing.xml”; created on Oct. 2, 2024, and 33,948 bytes in size, is hereby incorporated by reference.

### TECHNICAL FIELD

The present invention relates to a brain-metastatic lung cancer cell line and a use of Noggin as a marker for brain metastasis of lung cancer.

### BACKGROUND ART

Lung cancer is the leading cause of cancer-related deaths worldwide, with more than 1.8 million new cases diagnosed each year. The 5-year survival rate of lung cancer patients ranges from 4 to 17% depending on stage and regional differences, and despite advances in diagnostic and therapeutic techniques, most lung cancer patients are still diagnosed at advanced stages with a poor prognosis, and brain metastases are the main cause of this result. Metastasis, accounting for up to 90% of cancer-related deaths, occurs when epithelial cancer cells within the primary tumor undergo epithelial-to-mesenchymal transition (EMT), acquire motility and invasiveness of cancer cells, and allow cancer cells to spread to distant sites via the circulatory system. In addition, after completing mesenchymal-to-epithelial transition (MET), a metastatic tumor is established. Each cancer type has its own preferred organophilicity, and lung cancer generally leads to brain metastases in 40% of patients. However, the basic pathophysiological mechanism of brain metastasis in lung cancer is still elusive, highlighting the need for new biomarkers and potential treatment methods specifically targeting and inhibiting brain metastasis.

To confirm the genomic diversity between primary lung tumors and brain metastasis, focusing on identifying the most frequent genetic alterations, including EGFR, ALK and KRAS mutations, several studies have been conducted, and several recent studies have revealed that the presence of RET fusion in non-small cell lung cancer (NSCLC) patients is associated with an increased incidence of brain metastasis. Several potential lung cancer brain metastasis biomarkers have been identified in recent years, but most are not yet clinically approved.

Meanwhile, Noggin is a secreted protein belonging to the BMP antagonist family and has been shown to play a complex and situation-dependent role in cancer development. Noggin belongs to the TGF-β superfamily, and has been shown to inhibit the activity of BMPs, which are a protein group involved in various cellular processes such as cell proliferation, differentiation, and migration. Some studies have revealed that the growth of osteolytic prostate cancer lesions is inhibited by blocking BMP activity. On the other hand, in patients with gastric cancer, it is known that high Noggin expression is associated with poor prognosis, and the proliferation of gastric cancer cells is promoted by upregulating EGFR signaling. This shows that Noggin's cancer prevention and anticancer effects are complex and depend on the specific cancer type and situation. Accordingly, further research is needed to fully understand the role of Noggin in cancer development and explore its potential as a therapeutic target for cancer treatment.

Therefore, the present inventors established a brain metastatic lung cancer cell line that is specifically colonized in the brain by repeatedly transplanting the lung cancer cell line through the carotid artery and confirmed genetic changes associated with such brain colonization of the lung cancer cells, thereby confirming a potential marker for inhibiting brain metastasis of lung cancer.

### RELATED ART DOCUMENT

### Non-Patent Document

Thorac Cancer. 2020 November; 11(11): 3357-3364.

### DISCLOSURE

### Technical Problem

The present inventors established a brain metastatic lung cancer cell line having characteristics optimized for brain metastasis by repeatedly transplanting the lung cancer cell line through the carotid artery. In addition, as a result of confirming genetic changes associated with the brain colonization of lung cancer cells, it was confirmed that the expression of Noggin protein and its mRNA was decreased in the brain metastatic lung cancer cell line, which resulted in decreased cell motility and invasiveness and increased adhesion. Based on this, the present invention was completed.

Therefore, the present invention is directed to providing a brain metastatic lung cancer cell line, isolated from a lung cancer cell line colonized in the brain, and a method of preparing the same.

The present invention is also directed to providing a brain metastatic lung cancer animal model into which the brain metastatic lung cancer cell line is transplanted.

The present invention is also directed to providing an information providing method for diagnosing brain metastasis of lung cancer.

The present invention is also directed to providing a composition for diagnosing brain metastasis of lung cancer, which includes an agent for measuring the expression level of Noggin protein or its mRNA as an active ingredient.

The present invention is also directed to providing a pharmaceutical composition for inhibiting brain metastasis of lung cancer, which includes an expression or activation inducer of Noggin protein or its mRNA as an active ingredient.

The present invention is also directed to providing a method of screening a candidate material for inhibiting brain metastasis of lung cancer.

However, technical problems to be solved in the present invention are not limited to the above-described problems, and other problems which are not described herein will be fully understood by those of ordinary skill in the art from the following descriptions.

### Technical Solution

To achieve the above-described purposes, the present invention provides a brain metastatic lung cancer cell line, isolated from a lung cancer cell line colonized in the brain.

In one embodiment of the present invention, the lung cancer cell line may be a human lung cancer cell line A549, but the present invention is not limited thereto.

In another embodiment of the present invention, the brain metastatic lung cancer cell line may be A549-BM under Accession No. KCTC 15581BP, but the present invention is not limited thereto.

In still another embodiment of the present invention, the lung cancer cell line may be administered into the carotid artery and colonized in the brain, but the present invention is not limited thereto.

In yet another embodiment of the present invention, the brain metastatic lung cancer cell line may be reduced in motility and invasiveness and increased in adhesion during brain colonization, but the present invention is not limited thereto.

In yet another embodiment of the present invention, the brain metastatic lung cancer cell line may undergo mesenchymal-to-epithelial transition (MET), but the present invention is not limited thereto.

In yet another embodiment of the present invention, in the brain metastatic lung cancer cell line, the expression levels of one or more proteins selected from ZEB1, Vimentin, N-cadherin, and Twist1, or their mRNAs may be lower than those in a non-metastatic lung cancer cell line, but the present invention is not limited thereto.

In yet another embodiment of the present invention, in the brain metastatic lung cancer cell line, the expression level of E-cadherin protein or its mRNA may be higher than that in a non-metastatic lung cancer cell line, but the present invention is not limited thereto.

In addition, the present invention provides a method of preparing the brain metastatic lung cancer cell line, including the following steps:
(a) injecting an isolated lung cancer cell line into the carotid artery of an animal model;
(b) isolating brain tissue from the cell line-injected animal model, isolating the lung cancer cell line metastasized to the brain, and culturing the cell line; and
(c) injecting the isolated and cultured brain metastatic lung cancer cell line into the carotid artery of another animal model, and isolating the lung cancer cell line colonized in the brain.

In one embodiment of the present invention, in (a), the lung cancer cell line may be a human lung cancer cell line A549, but the present invention is not limited thereto.

In addition, the present invention provides a brain metastatic lung cancer animal model into which the brain metastatic lung cancer cell line is transplanted.

In one embodiment of the present invention, in the brain metastatic lung cancer animal model, the lung cancer cells may be colonized in the brain, but the present invention is not limited thereto.

In addition, the present invention provides an information providing method for diagnosing brain metastasis of lung cancer, which includes measuring the expression level of Noggin protein or its mRNA in a biological sample isolated from a subject.

In one embodiment of the present invention, the biological sample may be brain tissue-derived cells, but the present invention is not limited thereto.

In another embodiment of the present invention, when the expression level of Noggin protein or its mRNA is reduced compared to a control, the information providing method may further include determining that lung cancer has metastasized to the brain, but the present invention is not limited thereto.

In addition, the present invention provides a composition for diagnosing brain metastasis of lung cancer, which includes an agent for measuring the expression level of Noggin protein or its mRNA as an active ingredient.

In one embodiment of the present invention, the agent for measuring the protein expression level may be an antibody or aptamer specific to the protein, but the present invention is not limited thereto.

In another embodiment of the present invention, the agent for measuring the mRNA expression level may be a primer or probe specifically binding to the mRNA, but the present invention is not limited thereto.

In addition, the present invention provides a pharmaceutical composition for inhibiting brain metastasis of lung cancer, which includes an expression or activation inducer of Noggin protein or its mRNA as an active ingredient.

In one embodiment of the present invention, the expression or activation inducer of Noggin protein or its mRNA may be a recombinant vector containing Noggin gene, or Noggin protein, but the present invention is not limited thereto.

In another embodiment of the present invention, the pharmaceutical composition may inhibit brain colonization of lung cancer cells, but the present invention is not limited thereto.

In addition, the present invention provides a method of screening a candidate material for inhibiting the brain metastasis of lung cancer, which includes the following steps:
(a) treating the brain metastatic lung cancer cell line or the animal model with a candidate material;
(b) measuring the expression level of Noggin protein or its mRNA in the brain metastatic lung cancer cell line or animal model; and
(c) when the expression level of Noggin protein or its mRNA is increased compared to before the treatment with the candidate material, selecting the candidate material as a candidate material for inhibiting brain metastasis of lung cancer.

In addition, the present invention provides a method of inhibiting brain metastasis of lung cancer, which includes administering a composition including an expression or activation inducer of Noggin protein or its mRNA as an active ingredient into a subject in need thereof.

In addition, the present invention provides a use of a composition including an expression or activation inducer of Noggin protein or its mRNA as an active ingredient to inhibit the brain metastasis of lung cancer.

In addition, the present invention provides a use of a composition including an expression or activation inducer of Noggin protein or its mRNA as an active ingredient to prepare an agent for inhibiting the brain metastasis of lung cancer.

### Advantageous Effects

In the present invention, a brain metastatic lung cancer cell line with optimized characteristics for brain metastasis was established by repeatedly transplanting the lung cancer cell line through the carotid artery. In addition, in the brain metastatic lung cancer cell line, the expressions of Noggin protein and its mRNA were reduced, confirming that the motility and invasiveness of cells are reduced and adhesion increases. Therefore, the Noggin protein or its gene is expected to be useful as a biomarker for diagnosis of brain metastasis of lung cancer and its target therapy.

### DESCRIPTION OF DRAWINGS

FIG. 1A is a diagram schematically illustrating a process of establishing a brain metastatic lung cancer cell line according to one embodiment of the present invention.

FIG. 1B is a diagram confirming the morphology of A549-M0 and M2 cells according to one embodiment of the present invention.

FIG. 1C is a diagram confirming brain colonization in mice into which A549-M0 and M2 cells are injected through an in vivo bioluminescence image according to one embodiment of the present invention.

FIG. 2A is a diagram showing the growth rates of A549-M0 and M2 cells according to one embodiment of the present invention.

FIG. 2B is a diagram showing the results of analyzing clone production of A549-M0 and M2 cells according to one embodiment of the present invention.

FIG. 2C is a diagram showing the results of a soft agar colony assay for A549-M0 and M2 cells according to one embodiment of the present invention.

FIG. 2D is a diagram showing the results of a cell adhesion assay for A549-M0 and M2 cells according to one embodiment of the present invention.

FIG. 2E is a diagram showing the results of a Transwell migration assay for A549-M0 and M2 cells according to one embodiment of the present invention.

FIG. 2F is a diagram showing the results of a wound healing assay for A549-M0 and M2 cells according to one embodiment of the present invention.

FIG. 2G is a diagram showing the results of Western blotting, which confirm the protein expression levels of mesenchymal and epithelial markers in A549-M0 and M2 cells according to one embodiment of the present invention.

FIG. 2H is a diagram showing the results of qRT-PCR, which confirm the mRNA levels of mesenchymal and epithelial markers in A549-M0 and M2 cells according to one embodiment of the present invention.

FIG. 3A is a diagram showing the results of a Transwell migration assay for A549-M0 and M2 cells and astrocytes co-cultured therewith according to one embodiment of the present invention.

FIG. 3B is a diagram showing the results of a 3-well migration assay for A549-M0 and M2 cells and astrocytes co-cultured therewith according to one embodiment of the present invention.

FIG. 3C is a diagram showing the results of the invasion of A549-M0 and M2 cell spheroids into an astrocyte feeder layer according to one embodiment of the present invention.

FIG. 4A is a diagram showing the heatmap (left) showing the RNA sequence analysis data obtained from A549-M0 and M2 cells and the results of a gene set enrichment analysis (right) for genes differentially expressed in the A549-M0 and M2 cells according to one embodiment of the present invention.

FIG. 4B is a diagram showing the result of qRT-PCR that confirms the expression level of NOG mRNA in A549-M0 and M2 cells according to one embodiment of the present invention.

FIG. 4C is a diagram showing the results of qRT-PCR (left) and western blotting (right), which confirm the overexpression of Noggin in A549-M2 cells according to one embodiment of the present invention.

FIG. 4D is a diagram showing the results of a Transwell migration assay for A549-M0 and M2 cells, and Noggin-transfected M2 cells according to one embodiment of the present invention.

FIG. 4E is a diagram showing the results of a wound healing assay for Noggin-transfected M2 cells according to one embodiment of the present invention.

FIG. 4F is a diagram showing the result of a spheroid overlay analysis for Noggin-transfected M2 cells according to one embodiment of the present invention.

FIG. 4G is a diagram showing the result of qRT-PCR that confirms the expression level of NOG mRNA in Noggin siRNA-transfected M2 cells according to one embodiment of the present invention.

FIG. 4H is a diagram showing the result of a cell adhesion assay for Noggin siRNA-transfected M2 cells according to one embodiment of the present invention.

FIG. 4I is a diagram showing the result of a Transwell migration assay for Noggin siRNA-transfected M2 cells according to one embodiment of the present invention.

### MODES OF THE INVENTION

In one experimental example of the present invention, a brain metastatic lung cancer cell line was established by repeatedly performing a process of injecting an A549 human lung cancer cell line into a mouse's carotid artery and isolating brain-colonized lung cancer cells twice, and named A549-M2 (refer to Experimental Example 1).

In another experimental example of the present invention, it was confirmed that brain-colonized lung cancer cells (A549-M2) exhibit a lower growth rate, less colony formation, higher cell adhesion, and lower motility than mother A549 lung cancer cells (A549-M0). In addition, it was confirmed that, compared to M0 cells, in M2 cells, the protein expression levels of mesenchymal markers such as ZEB1, Vimentin, N-cadherin, and Twist1 are relatively lower, and the protein expression level of epithelial marker E-cadherin is relatively higher, identifying that metastatic lung cancer cells can undergo mesenchymal-to-epithelial transition (MET) after brain colonization (refer to Experimental Example 2).

In still another experimental example of the present invention, as a result of confirming the interaction between lung cancer cells and astrocytes during brain colonization, it was confirmed that M0 cells interact with astrocytes and are mixed well therewith, compared to M2 cells. From this, it was seen that metastatic lung cancer cells reduce invasiveness after brain colonization (refer to Experimental Example 3).

In yet another experimental example of the present invention, it was confirmed that, in M2 cells, the mRNA expression of Noggin is downregulated, and Noggin overexpression recovers suppressed migration of M2 cells. In addition, it was confirmed that, in the control A549 cells, when Noggin gene was knocked down using siRNA, cell adhesion was promoted, and cell migration was suppressed, and thus the decrease in Noggin during brain colonization can inhibit migration and invasion of metastatic lung cancer cells (refer to Experimental Example 4).

Hereinafter, the present invention will be described in detail.

The present invention provides a brain metastatic lung cancer cell line, isolated from a lung cancer cell line colonized in the brain.

“Cell line” used herein refers to an individual cell line when cells are isolated, pure-cultured, and then subcultured. Here, the cell line may be distinguished from another cell line by genetic characteristics, and the characteristics of the original cells are maintained after subculture.

In the present invention, the lung cancer cell line may be human lung cancer cell line A549, but the present invention is not limited thereto.

In the present invention, a brain metastatic lung cancer cell line was established by repeatedly performing a process of injecting the human lung cancer cell line A549 into a mouse's carotid artery and isolating the brain-colonized lung cancer cells twice, and named A549-BM (“A549-M2” is also used with the same meaning in the specification), and deposited at the Korean Collection for Type Culture (KCTC) on Aug. 31, 2023, and assigned Accession No. KCTC 15581BP.

“Lung cancer” used herein refers to a malignant tumor generated in the lungs, and may occur in the lungs themselves (primary lung cancer) or occur due to metastasis of cancer that has occurred in another organ to the lungs. Primary lung cancer is classified into non-small cell lung cancer and small cell lung cancer based on the size and shape of the cancer cells. About 15% of lung cancers are small cell lung cancer, and the remaining 85% are non-small cell lung cancer, including adenocarcinomas, squamous cell carcinomas, and large cell carcinomas.

“Metastasis” used herein means that cancer cells leave the primary organ and move to another organ. Cancer spreads to other parts of the body in two ways: cancer tissue grows from the primary tumor and directly invades surrounding organs; and distant metastasis occurs through blood vessels or lymphatic vessels to distant organs.

“Brain colonization” used herein refers to the colonization of lung cancer cell lines in brain tissue or brain cells without tissue invasion or damage.

In the present invention, the lung cancer cell line may be injected into the carotid artery and colonized in the brain, and the brain metastatic lung cancer cell line may be reduced in motility and invasiveness and increased in adhesion during brain colonization, but the present invention is not limited thereto.

In the present invention, the brain metastatic lung cancer cell line may undergo mesenchymal-to-epithelial transition (MET), but the present invention is not limited thereto.

In the present invention, “mesenchymal-to-epithelial transition (MET)” is the reverse process of epithelial mesenchymal transition (EMT), and is known to be involved in stability and settlement of metastatic cancer cells to form colonies (colonization) in cells that have metastasized, and to obtain the characteristics of epithelial cells and the properties to fuse with distant organs. EMT, which is the reverse process of MET, is considered important in cancer metastasis.

In the present invention, in the brain metastatic lung cancer cell line, the expression level of one or more proteins selected from the group consisting of ZEB1, Vimentin, N-cadherin, and Twist1 or its mRNA may be lower than a level in a lung cancer cell line that is not metastasized in the brain, but the present invention is not limited thereto.

In the present invention, in the brain metastatic lung cancer cell line, the expression level of E-cadherin protein or its mRNA may be higher than levels in the non-brain metastatic lung cancer cell line, but the present invention is not limited thereto.

In addition, the present invention provides a method of preparing the brain metastatic lung cancer cell line, which includes the following steps:
(a) injecting an isolated lung cancer cell line into the carotid artery of an animal model;
(b) isolating a lung cancer cell line which has been isolated from brain tissue of the cell line-injected animal model and metastasized to the brain and then culturing it; and
(c) injecting the isolated and cultured brain metastatic lung cancer cell line into the carotid artery of another animal model and then isolating the lung cancer cell line colonized in the brain.

In the present invention, in (a), the lung cancer cell line may be a human lung cancer cell line A549, but the present invention is not limited thereto.

In the present invention, in (a), the lung cancer cell line may be injected into the carotid artery of an animal model, and then the animal model may be euthanized after 6 to 10 weeks, 6 to 9 weeks, 6 to 8 weeks, 7 to 10 weeks, 7 to 9 weeks, 7 to 8 weeks, 8 to 10 weeks, 8 to 9 weeks, or 8 weeks. Subsequently, in (b), the brain tissue may be isolated, but the present invention is not limited thereto.

In the present invention, in (b), the separating of the brain metastatic lung cancer cell line may include cutting the isolated brain tissue, precipitating it by mixing with trypsin, and filtering the precipitate through a cell filter, but the present invention is not limited thereto.

In the present invention, the culture in (b) may be performed at 33 to 40° C., 33 to 39° C., 33 to 38° C., 33 to37° C., 35 to40° C., 35 to39° C., 35 to38° C., 35 to37° C., or 37° C., but the present invention is not limited thereto.

In the present invention, in the preparation of the brain metastatic lung cancer cell line, a step of isolating and culturing the brain colonized lung cancer cell line and injecting it again to the carotid artery of another animal model may be performed repeatedly, for example, two to three times, two times, or three times, but the present invention is not limited thereto.

In addition, the present invention provides a brain metastatic lung cancer animal model into which the brain metastatic lung cancer cell line is transplanted.

In the present invention, in the brain metastatic lung cancer animal model, lung cancer cells may be colonized in the brain, but the present invention is not limited thereto.

In the present invention, “animal model” refers to an animal with a similar type of disease to a human disease. The significance of disease model animals in the study of human diseases is due to the physiological or genetic similarity between humans and animals. In disease research, biomedical disease model animals provide research materials for various causes and pathogenesis, and diagnosis of diseases, and through research on disease model animals, disease-associated genes are identified, and the interaction between genes may be understood. In addition, through actual efficacy and toxicity tests for a developed new drug candidate, basic materials for determining the possibility of commercialization may be obtained.

In the present invention, the animal is any mammal other than a human. The animal may be an animal of any age, including an embryo, a fetus, a newborn, and an adult. Animals for use in the present invention may be available, for example, from commercial sources. Such animals include, but are not limited to, laboratory animals or other animals, rabbits, rodents (e.g., mice, rats, hamsters, gerbils, and guinea pigs), cattle, sheep, pigs, goats, horses, dogs, cats, birds (e.g., chickens, turkeys, ducks, and geese), and primates (e.g., chimpanzees, monkeys, and Rhesus macaques). According to one embodiment or experimental example of the present invention, the animal may be a mouse, but the present invention is not limited thereto.

In addition, the present invention provides a method of preparing a brain metastatic lung cancer animal model, which includes injecting the brain metastatic lung cancer cell line into the carotid artery of an animal model.

In addition, the present invention provides an information providing method for diagnosing brain metastasis of lung cancer and/or a method of diagnosing brain metastasis of lung cancer, which includes measuring the expression level of Noggin protein or its mRNA in a biological sample isolated from a subject.

In addition, the present invention provides an information providing method for predicting the risk of brain metastasis of lung cancer and/or a method of predicting the risk of brain metastasis of lung cancer, which includes measuring the expression level of Noggin protein or its mRNA in a biological sample isolated from a subject.

In the present invention, “Noggin” is a protein involving the development of many body tissues including nerve tissue, muscles and bones, and in the case of humans, is encoded by NOG gene. In the present invention, the Noggin may comprise or consist of the amino acid sequence having a sequence homology of 70% or more, 75% or more, 80% or more, 85% or more, 90% or more, 95% or more, 98% or more, 70 to 100%, 75 to 100%, 80 to 100%, 85 to 100%, 90 to 100%, 95 to 100%, 98 to 100%, 90%, 91%, 92%, 93%, 94%, 95%, 96%, 97%, 98%, 99%, or 100% with the amino acid sequence of GenBank: AAA83259.1 (SEQ ID NO: 1), but the present invention is not limited thereto.

In the present invention, the biological sample is any sample from which the expression or expression level of Noggin protein or its mRNA in the body can be confirmed, and preferably includes all samples, blood, plasma, serum, bone marrow, tissue, cells, saliva, sputum, peritoneal fluid, hair, nasal fluid, urine, and feces, obtained in biopsy, and according to one embodiment of the present invention, the biological sample may be brain tissue-derived cells, or may be any sample containing Noggin protein or its mRNA without limitation.

In the present invention, the above method may further include, when the expression level of Noggin protein or its mRNA is reduced compared to the control, determining that lung cancer is metastasized to the brain; or predicting a high risk of lung cancer metastasized to the brain, but the present invention is not limited thereto.

In the present invention, the control may include both a normal control or a lung cancer cell line without brain metastasis (primary lung cancer cell line), but the present invention is not limited thereto.

In the present invention, examples of the subject may include humans or non-human primates, and mammals such as mice, rats, dogs, cats, horses, and cattle, and may be a patient who will be diagnosed with brain metastasis of lung cancer, but the present invention is not limited thereto.

In the present invention, the protein expression level may be measured using one or more methods selected from mass spectrometry, western blotting, enzyme-linked immunoassay (ELISA), capture-ELISA, inhibition or competition assay, sandwich assay, radioimmunoassay ( [symbol] , [symbol] , [symbol] ), radial immunodiffusion, Ouchterlony immunodiffusion, Rocket immunoelectrophoresis, immunohistochemical staining, immunofluorescence staining, immunoaffinity purification, radioimmunoprecipitation, immunoprecipitation assay, complement fixation assay, flow cytometry (FACS), and protein chip, but the present invention is not limited thereto.

In the present invention, the mRNA expression level may be measured using one or more methods selected from the group consisting of polymerase chain reaction (PCR), reverse transcription polymerase chain reaction (RT-PCR), competitive RT-PCR, quantitative real-time polymerase chain reaction (qRT-PCR), real-time PCR, quantitative PCR, quantitative real-time PCR, RNase protection assay (RPA), Northern blotting, and DNA chip, but the present invention is not limited thereto.

In addition, the present invention provides a composition and/or kit for diagnosing brain metastasis of lung cancer, which includes an agent for measuring the expression level of Noggin protein or its mRNA as an active ingredient.

In the present invention, “diagnosis” refers to confirming the presence or characteristic of the pathological state. For the purpose of the present invention, diagnosis is not limited to determining whether brain metastasis of lung cancer occurs, but includes determining the degree of brain metastasis of lung cancer.

In the present invention, “measurement” includes both detecting and confirming the presence (expression) of a target material, and detecting and confirming a change in the presence (expression level) of a target material. The measurement may be performed by a qualitative method (analysis) or quantitative method without limitation. For the determination of the presence of one or more selected from the group consisting of Noggin protein and its mRNA, types of qualitative and quantitative methods are well known in the art, the experimental methods described in the specification are well known in the art, and the experimental methods described in the specification are included.

In the present invention, “expression” refers to a process of producing a polypeptide from a structural gene. This process includes the transcription of a gene into mRNA, and the translation of this mRNA into polypeptide(s).

In the present invention, the agent for measuring the protein expression level may be an antibody or aptamer specific to the protein, but the present invention is not limited thereto.

In the present invention, “antibody” refers to a protein molecule that is presented to an antigenic site and binds specifically thereto. Antibodies may be produced by methods commonly used in the art, such as a fusion method, a recombinant DNA method, and a phage antibody library method. In some embodiments, the antibody or fragment thereof may be derived from a different organism such as a human, mouse, rat, hamster, rabbit, or camel, and may be, for example, a monoclonal or polyclonal antibody, an immunologically active fragment, an antibody heavy chain, a humanized antibody, an antibody light chain, a genetically engineered single-chain Fv molecule, or a chimeric antibody. In the present invention, the antibody is not limited to a specific type of antibody as long as it can detect the protein of the present invention.

In the present invention, “aptamer” refers to a single-stranded nucleic acid (DNA, RNA, or a modified nucleic acid) having a stable tertiary structure and able to bind to a target molecule with high affinity and specificity, and aptamers can be developed for various target materials (proteins, sugars, dyes, DNA, metal ions, cells, etc.) using a method called systematic evolution of ligands of exponential enrichment (SELEX).

In the present invention, the agent for measuring an mRNA expression level may be a primer or probe that specifically binds to mRNA, but the present invention is not limited thereto.

In the present invention, “primer” is a short single strand oligonucleotide that acts as a starting point of DNA synthesis. A primer specifically binds to a polynucleotide, which is a template, in an appropriate buffer under an appropriate temperature condition, and DNA polymerase allows a nucleoside triphosphate having a complementary base to the template DNA to be linked to the primer, resulting in synthesizing DNA. A primer generally consists of the sequence of 15 to 30 bases, and a melting temperature (Tm) at which the bases bind to the template strand varies depending on the base composition and length of the primer. The sequence of a primer does not need to have a sequence that is perfectly complementary to a part of the base sequence of the template, but it is sufficient as long as the primer has a length and complementarity suitable for the purpose of measuring the amount of mRNA by amplifying a specific section of mRNA or cDNA through DNA synthesis. Therefore, in the present invention, a primer pair may be easily designed with reference to the base sequence of mRNA or its cDNA. The primers for amplification are composed of a set (pair) that complementarily binds to the template (or sense) and the opposite end (antisense) of a specific section of mRNA to be amplified.

In the present invention, “probe” refers to a fragment of a polynucleotide, such as RNA, DNA, or miRNA, with a length of at least several to a maximum of hundreds of base pairs that can specifically bind to mRNA, complementary DNA (cDNA), or DNA of a specific gene, and may be labeled to confirm the presence or absence and the expression level of the binding target mRNA or cDNA. Conditions for probe selection and hybridization may be appropriately selected according to techniques known in the art. The diagnosis method may include detection methods based on the hybridization of nucleic acids, such as Southern blotting, and a probe may be provided having previously been bound to a substrate of a DNA chip in a method using a DNA chip.

In the present invention, the primer or probe may be chemically synthesized using a phosphoramidite solid support synthesis method or other widely known methods. In addition, the primer or probe may be modified in various forms by a method known in the art without interfering with hybridization with a polynucleotide, which becomes a target to be detected. Examples of such modifications include methylation, capping, substitution with one or more homologs of a natural nucleotide, modification between nucleotides, such as uncharged linkers (e.g., methyl phosphonate, phosphotriester, phosphoramidate, carbamate, etc.), or charged linkers (e.g., phosphorothioate, phosphorodithioate, etc.), and binding of a labeling material using fluorescence or an enzyme.

In the present invention, the primer or probe is not limited to a specific sequence as long as it can measure the mRNA expression.

In the present invention, the kit for diagnosing brain metastasis of lung cancer, including an agent for measuring the expression level of Noggin protein or its mRNA as an active ingredient, may include instructions on the information providing method for diagnosing brain metastasis of lung cancer and/or predicting the risk of brain metastasis of lung cancer according to the present invention, but the present invention is not limited thereto.

In the present invention, “kit” refers to a tool that can diagnose or predict brain metastasis of lung cancer, including an agent for measuring the expression of the protein or its mRNA. The kit of the present invention may include other components, compositions, solutions, or devices, which are conventionally required for methods for measuring or detecting them, in addition to the above-described agent. Here, a material for detecting the expression of the protein or its mRNA may be applied one or more times without limit, and there is no limit to the timing of the application of each material, and the application of each material may be done simultaneously or at different times.

In the present invention, the kit may include a container; instructions; and an agent for measuring the expression of the protein or its mRNA. The container may serve to package the agent, and also serve to store and fix it. The material of the container may be provided in a form such as a bottle, a tub, a sachet, an envelope, a tube, or an ampoule, and may be formed partially or entirely from plastic, glass, paper, foil, or wax. The container may be equipped with a completely or partially removable closure, which may initially be part of the container or may be attached to the container by mechanical, adhesive, or other means, or may be equipped with a stopper through which a needle can access the contents. The kit may include an external package, and the external package may include instructions on the use of components.

In addition, the present invention provides a method of providing information for determining/analyzing whether a subject is susceptible to diagnosing brain metastases of lung cancer or predicting the risk of brain metastases of lung cancer, when the expression levels of Noggin protein or its mRNA are measured in biological samples isolated from a subject suspected of brain metastasis of lung cancer and a control, the difference in expression level increases (two-fold or more) indicate that the subject has brain metastasis of lung cancer.

In addition, the present invention provides a pharmaceutical composition for inhibiting brain metastasis of lung cancer, which includes an expression or activation inducer of Noggin protein or its mRNA as an active ingredient.

In the present invention, the expression or activation inducer of Noggin protein or its mRNA is not limited to any type of material that induces or increases the expression or activity of Noggin protein or is mRNA, and may include, for example, a recombinant vector containing Noggin gene, or Noggin protein.

In the present invention, “recombinant vector” may be used to refer to the expression vector of a target polypeptide, which can express the target polypeptide in appropriate host cells with high efficiency when an encoded gene of the target polypeptide to be expressed is operably linked, and the recombinant vector can be expressed in host cells. The host cells are preferably eukaryotic cells, and depending on the type of host cells, they may appropriately select expression regulatory sequences such as a promoter, a terminator, an enhancer, etc., and sequences for membrane targeting or secretion, and may combine them in various ways according to the purpose.

In the present invention, the recombinant vector may be an expression vector of Noggin protein, which can clone human NOG cDNA in the vector. There are no limitations on the type of vector, the method of preparing a recombinant vector, or a method of transducing the recombinant vector, which are used herein, and methods known in the art may be used.

In the present invention, the pharmaceutical composition may inhibit the brain colonization of lung cancer cells, but the present invention is not limited thereto.

The pharmaceutical composition according to the present invention may further include appropriate carriers, excipients, and diluents, which are generally used in the preparation of pharmaceutical compositions. The excipient may be, for example, one or more selected from the group consisting of a diluent, a binder, a disintegrant, a lubricant, an adsorbent, a humectant, a film-coating material, and a controlled-release additive.

The pharmaceutical composition according to the present invention may be formulated in the form of a powder, a granule, a sustained-release granule, an enteric granule, a liquid, an ophthalmic solution, an elixir, an emulsion, a suspension, a spirit, a troche, aromatic water, a lemonade, a tablet, a sustained-release tablet, an enteric tablet, a sublingual tablet, a hard capsule, a soft capsule, a sustained-release capsule, an enteric capsule, a pill, a tincture, a soft extract, a dry extract, a fluid extract, an injection, a capsule, a perfusate, a plaster, a lotion, a paste, a spray, an inhalant, a patch, a sterile injection, or an external preparation such as an aerosol according to a conventional method, and the external preparation may be formulated in a cream, a gel, a patch, a spray, an ointment, a plaster, a lotion, a liniment, a paste or a cataplasma.

The carriers, excipients, and diluents, which can be included in the pharmaceutical composition according to the present invention, may include lactose, dextrose, sucrose, an oligosaccharide, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oil.

The pharmaceutical composition according to the present invention may be prepared using a diluent or an excipient such as a filler, a thickening agent, a binder, a wetting agent, a disintegrant, and a surfactant, which are commonly used.

Additives for the tablet, powder, granule, capsule, pill and troche according to the present invention may include excipients such as corn starch, potato starch, wheat starch, lactose, sucrose, glucose, fructose, di-mannitol, precipitated calcium carbonate, synthetic aluminum silicate, calcium monohydrogen phosphate, calcium sulfate, sodium chloride, sodium bicarbonate, purified lanolin, microcrystalline cellulose, dextrin, sodium alginate, methylcellulose, carboxymethylcellulose, sodium carboxymethylcellulose, kaolin, urea, colloidal silica gel, hydroxypropyl starch, hydroxypropyl methyl cellulose (HPMC) 1928, HPMC 2208, HPMC 2906, HPMC 2910, propylene glycol, casein, calcium lactate and Primojel; binders such as gelatin, gum arabic, ethanol, agar powder, cellulose acetate phthalate, carboxymethyl cellulose, carboxymethyl cellulose calcium, glucose, purified water, sodium caseinate, glycerin, stearic acid, sodium carboxymethylcellulose, sodium methylcellulose, methylcellulose, microcrystalline cellulose, dextrin, hydroxycellulose, hydroxypropyl starch, hydroxymethylcellulose, purified shellac, starch powder, hydroxypropylcellulose, hydroxypropylmethylcellulose, polyvinyl alcohol and polyvinylpyrrolidone; disintegrants such as hydroxypropylmethylcellulose, corn starch, agar powder, methylcellulose, bentonite, hydroxypropyl starch, sodium carboxymethylcellulose, calcium citrate, sodium lauryl sulfate, silicic anhydride, 1-hydroxypropyl cellulose, dextran, an ion exchange resin, polyvinyl acetate, formaldehyde-treated casein and gelatin, alginic acid, amylose, guar gum, sodium bicarbonate, polyvinylpyrrolidone, calcium phosphate, gelled starch, gum arabic, amylopectin, pectin, sodium polyphosphate, ethyl cellulose, sucrose, magnesium aluminum silicate, a di-sorbitol solution and light anhydrous silicic acid; and lubricants such as calcium stearate, magnesium stearate, stearic acid, hydrogenated vegetable oil, talc, lycopodium powder, kaolin, petrolatum, sodium stearate, cacao butter, sodium salicylate, magnesium salicylate, polyethylene glycol (PEG) 4000, PEG 6000, liquid paraffin, hydrogenated soybean oil (Lubri wax), aluminum stearate, zinc stearate, sodium lauryl sulfate, magnesium oxide, Macrogol, synthetic aluminum silicate, silicic anhydride, a higher fatty acid, a higher alcohol, silicone oil, paraffin oil, polyethylene glycol fatty acid ether, starch, sodium chloride, sodium acetate, sodium oleate, dl-leucine, and light anhydrous silicic acid.

Additives for the liquid according to the present invention may be water, diluted hydrochloric acid, diluted sulfuric acid, sodium citrate, monostearate sucrose, polyoxyethylene sorbitol fatty acid esters (Tween esters), polyoxyethylene monoalkylethers, lanolin ethers, lanolin esters, acetic acid, hydrochloric acid, acetic acid, hydrochloric acid, aqueous ammonia, ammonium carbonate, potassium hydroxide, sodium hydroxide, prolamine, polyvinylpyrrolidone, ethyl cellulose, and sodium carboxymethylcellulose.

The syrup according to the present invention may include a solution of white sugar, a different type of sugar, or a sweetener, and if necessary, a flavoring agent, a colorant, a preservative, a stabilizer, a suspending agent, an emulsifier, or a thickener.

For the emulsion according to the present invention, distilled water may be used, and if necessary, an emulsifier, a preservative, a stabilizer, and a flavoring agent may be used.

The suspension according to the present invention may include a suspending agent such as acacia, tragacanth, methylcellulose, carboxymethylcellulose, sodium carboxymethylcellulose, microcrystalline cellulose, sodium alginate, hydroxypropylmethyl cellulose (HPMC), HPMC 1828, HPMC 2906, or HPMC 2910, and if necessary, a surfactant, a preservative, a stabilizer, a colorant, or a flavoring agent.

The injections according to the present invention may include a solvent such as injectable sterile water, 0.9% sodium chloride for injection, Ringer's solution, dextrose for injection, dextrose+sodium chloride for injection, PEG, lactated Ringer's solution, ethanol, propylene glycol, non-volatile oil-sesame oil, cottonseed oil, peanut oil, soybean oil, corn oil, ethyl oleate, isopropyl myristic acid or benzene benzoate; a solubilizing agent such as sodium benzoate, sodium salicylate, sodium acetate, urea, urethane, monoethylacetamine, butazolidine, propylene glycol, Tween, nicotinamide, hexamine or dimethylacetamide; a buffer such as a weak acid and a salt thereof (acetic acid and sodium acetate), a weak base and a salt thereof (ammonia and ammonium acetate), an organic compound, a protein, albumin, peptone, or gums; an isotonic agent such as sodium chloride; a stabilizer such as sodium bisulfite (NaHSO₃), carbon dioxide gas, sodium metabisulfite (Na₂S₂O₃), sodium sulfite (Na₂SO₃), nitrogen gas (N₂) or ethylenediamine tetraacetic acid; an antioxidant such as sodium bisulfide 0.1%, sodium formaldehyde sulfoxylate, thiourea, disodium ethylenediaminetetraacetate or acetone sodium bisulfite; an analgesic such as benzyl alcohol, chlorobutanol, procaine hydrochloride, glucose or calcium gluconate; or a suspending agent such as sodium CMC, sodium alginate, Tween 80 or aluminum monostearate.

The suppositories according to the present invention may include a base such as cacao butter, lanolin, Witepsol, polyethylene glycol, glycerogelatin, methyl cellulose, carboxymethylcellulose, a mixture of stearate and oleate, Subanal, cottonseed oil, peanut oil, palm oil, cacao butter+cholesterol, lecithin, Lanette wax, glycerol monostearate, Tween or Span, Imhausen, monolene (propylene glycol monostearate), glycerin, Adeps solidus, Buytyrum Tego-G, Cebes Pharma 16, hexalide base 95, Cotomar, Hydrokote SP, S-70-XXA, S-70-XX75 (S-70-XX95), Hydrokote 25, Hydrokote 711, Idropostal, Massa estrarium, (A, AS, B, C, D, E, I, T), Mass-MF, Masupol, Masupol-15, neosuppostal-N, paramount-B, supposiro (OSI, OSIX, A, B, C, D, H, L), suppository base IV types (AB, B, A, BC, BBG, E, BGF, C, D, 299), Suppostal (N, Es), Wecoby (W, R, S, M, Fs), or a Tegester triglyceride base (TG-95, MA, 57).

Examples of solid preparations for oral administration are tablets, pills, powders, granules, and capsules, and such solid preparations are prepared by mixing at least one excipient, such as starch, calcium carbonate, sucrose, lactose, or gelatin, with the composition. Aside from simple excipients, lubricants such as magnesium stearate and talc are further used.

Examples of liquid formulations for oral administration may be a suspension, a liquid for internal use, an emulsion, and a syrup, and may include a generally used simple diluent such as water or liquid paraffin, as well as various types of excipients, for example, a wetting agent, a sweetener, a flavoring agent and a preservative. Formulations for parenteral administration may include sterilized aqueous solutions, non-aqueous solvents, suspensions, emulsions, freeze-dried formulations, or suppositories. As the non-aqueous solvent or suspension, propylene glycol, polyethylene glycol, a vegetable oil such as olive oil, or an injectable ester such as ethyl oleate may be used.

The pharmaceutical composition of the present invention is administered at a pharmaceutically effective amount. “Pharmaceutically effective amount” used herein refers to an amount sufficient for treating a disease at a reasonable benefit/risk ratio applicable for medical treatment, and an effective dosage may be determined by parameters including the type of a patient's disease, severity, drug activity, sensitivity to a drug, administration time, an administration route and an excretion rate, the duration of treatment and drugs simultaneously used, and other parameters well known in the medical field.

The pharmaceutical composition of the present invention may be administered separately or in combination with other therapeutic agents, and may be sequentially or simultaneously administered with a conventional therapeutic agent, or administered in a single or multiple dose(s). In consideration of all of the above-mentioned parameters, it is important to achieve the maximum effect with the minimum dose without side effects, and such a dose may be easily determined by one of ordinary skill in the art.

The pharmaceutical composition of the present invention may be administered to a subject via various routes. All administration routes may be contemplated, and the pharmaceutical composition of the present invention may be administered by, for example, oral administration, subcutaneous injection, intraperitoneal administration, intravenous, intramuscular or intrathecal injection, sublingual administration, buccal administration, rectal insertion, vaginal insertion, ocular administration, ear administration, nasal administration, inhalation, spraying through the mouth or nose, skin administration, or transdermal administration.

The pharmaceutical composition of the present invention is determined according to the type of drug as an active ingredient as well as various related parameters such as a disease to be treated, an administration route, a patient's age, sex, and body weight, and the severity of a disease.

“Subject” used herein refers to a subject in need of treatment for a disease, and more specifically, a human or a non-human primate, or a mammal such as a mouse, a rat, a dog, a cat, a horse, or a cow.

“Administration” used herein refers to providing the given composition of the present invention to a subject by any suitable method.

In the present invention, “treatment” refers to all actions that alleviate or beneficially change a target disease and associated metabolic abnormalities thereof by administration of the pharmaceutical composition according to the present invention.

In addition, the present invention provides a method of screening a candidate material for inhibiting brain metastasis of lung cancer, which includes the following steps:
(a) treating the brain metastatic lung cancer cell line or the brain metastatic lung cancer animal model with a candidate material;
(b) measuring the expression level of Noggin protein or its mRNA in the brain metastatic lung cancer cell line or animal model; and
(c) when the expression level of Noggin protein or its mRNA is increased compared to that before the treatment with the candidate material, selecting the candidate material as a candidate material for inhibiting brain metastasis of lung cancer.

In the present invention, “candidate material” means an unknown material that is used in screening to confirm the expression or expression level of Noggin protein or its mRNA in the brain metastatic lung cancer cell line or brain metastatic lung cancer animal model, for example, one or more selected from the group consisting of a nucleotide, DNA, RNA, an amino acid, an aptamer, a protein, a compound, a natural material, and a natural extract, but the present invention is not limited thereto.

In the present invention, the treating of the candidate material means, after adding a candidate material to the cell line, culturing the cell line for a predetermined time, or orally administering or injecting the candidate material to an animal model, but the present invention is not limited thereto.

In addition, the present invention provides a method of inhibiting brain metastasis of lung cancer, which includes administering a composition including an expression or activation inducer of Noggin protein or its mRNA as an active ingredient to a subject in need thereof.

In addition, the present invention provides a use of a composition including an expression or activation inducer of Noggin protein or its mRNA as an active ingredient to inhibit brain metastasis of lung cancer.

In addition, the present invent ion provides a use of a composition including an expression or activation inducer of Noggin protein or its mRNA as an active ingredient to prepare an agent for inhibiting brain metastasis of lung cancer.

When the term “including” or “comprising” is used herein, it does not exclude other components unless specifically stated otherwise and other components may be further included. The term “step” or “stage” of something used throughout the present invention does not mean a step for something.

Hereinafter, preferred examples are presented to allow the present invention to be better understood. However, the following examples are merely provided to more easily understand the present invention, and the content of the present invention is not limited by the following examples.

### EXAMPLES

### Example 1. Mouse Experiment

All mouse studies were submitted to and approved by the Catholic University of Korea Institutional Animal Care and Use Committee (IACUC; CUMC-2016-0273-01), and A549 was purchased from ATCC. A cell line was authenticated using STR profiling with the Powerplex 18D system (Promega, Madison, WI, USA) by COSMOgenetech (Daejeon, Korea) within the last three years. In addition, the mice were cared for and euthanized according to the standards established by IACUC.

Specifically, after respiratory anesthesia with isoflurane, the mouse (BALB/c nude) was placed in a supine position. For blunt dissection of cervical tissue under a stereomicroscope, a 1.0 cm-long skin incision was made in the center of the neck to expose the pulsating right common carotid artery. The proximal part of the common carotid artery was ligated using 7-0 suture, and the distal part was wrapped without ligation. The right external carotid artery was also ligated. To make a puncture in the common carotid artery, a cannula tip was pushed into the internal carotid artery using a 31-gauge needle. Subsequently, after injecting 10 μL of A549 cell suspension (100 cell/μL), the syringe was removed and the right common carotid artery was ligated using a distal suture. After skin closure, the postoperative activity of the mouse was monitored, and the mouse was euthanized about 8 weeks after cell transplantation.

### Example 2. Brain Colonization of Lung Cancer Cells

After confirming brain metastases by bioluminescence imaging, fresh brain tissue was cut into fine pieces (about 1 mm³) and immersed in 2.5% trypsin (20 mL per 1 g of tissue, Welgene, Gyeongsan, Korea) with slow but continuous mixing. Then, the sample was gradually filtered through a cell strainer with a pore size of 40 μm (SPL Life Sciences, Pocheon, Korea). The single cell suspension was diluted with 10% fetal bovine serum (Corning; Corning, NY, USA)-supplemented DMEM (Welgene), and isolated cancer cells (A549-M1) were put into a T25 culture flask (SPL Life Sciences) and cultured under conditions of 37° C. and 5% Co₂. The A549-M1 cells were repeatedly injected into a mouse via the carotid artery to isolate the A549-M2 cells colonized in the brain, and the cells were established as above.

### Example 3. Cell Culture

A549-M0 and A549-M2 human lung cancer cells were cultured in RPMI 1640 (Welgene), and immortalized human astrocytes (abm; Richmond, BC, Canada) and 293T were cultured in 10% fetal bovine serum-supplemented DMEM (Welgene) under conditions of 37° C. and 5% CO₂.

For actin staining, cells were cultured on coverslips coated with 0.1 mg/mL collagen, fixed with 4% formaldehyde, and stained with Alexa Fluor 594-phalloidin (Invitrogen, Carlsbad, CA, USA) and DAPI (5 μg/mL) according to the manufacturers' protocols. Cell counts were calculated using a LUNA automatic cell counter (Logos Biosystems, Anyang, Korea).

For a clonogenic assay, cells (200 cells/well) were seeded in a 6-well plate, and after 6 days, stained with 0.1% crystal violet.

For soft agar colony assays, cells (5×10⁴ cells/well) were suspended in 0.3% agarose and inoculated into a 6-well plate on which 0.8% agarose was layered, and after three weeks, colonies were stained with 0.5 mg/mL of nitro blue tetrazolium.

For wound healing assay, cells (6×10⁵ cells/well) were inoculated into a 6-well plate. Upon reaching confluence, the cell monolayer was scraped in a straight line using a pipette tip and incubated with 1 μg/mL of mitomycin C for 48 hours. A wound area was measured using ImageJ (NIH, Bethesda, MD, USA).

For Noggin overexpression, human NOG cDNA of a pDNR-LIB vector (#hMU013799) was obtained from the Korean Human Gene Bank (Genomic medicine Institute, KRIBB, Korea). NOG cDNA was cloned in a pLVX-Neo vector, and transduced into A549-M2 cells by lentivirus infection. Human NOG siRNA was purchased from Bioneer (Daejeon, Korea), and temporarily transduced into lung cancer cells using the TransIT-X2 Dynamic Delivery System (Mirus Bio, Madison, WI, USA). The NOG siRNA sequence is show in Table 1 below.

TABLE 1

| NOG | | SEQ |
|---|---|---|
| siRNAs | Sequence of validated siRNAs | ID NO: |
| #1 | AGA GAG ACU UAU UCU GGU U | 2 |
| | AAC CAG AAU AAG UCU CUCU | 3 |
| #2 | CAU UCU UCG GAA AGU GUU U | 4 |
| | AAA CAC UUU CCG AAG AAU G | 5 |
| #3 | GAA GCU GCG GAG GAA GUU A | 6 |
| | UAA CUU CCU CCG CAG CUU C | 7 |

For fluorescence labeling, a pLVX-Puro/EGFP vector (green fluorescence) was transduced into A549-M0 and A549-M2 cells, and a pCDH-CMV-mCherry-EF1 Hygro vector (red fluorescence, Oskar Laur, Addgene plasmid #129440) was transduced into astrocytes. All experiments were performed using mycoplasma-free cells.

### Example 4. Transwell Migration Assay

Cells (1×10⁵ cells/well) were seeded in a Transwell insert (Falcon Cell Culture Inserts, 8-μm pore, Corning) and cultured for 24 hours to allow the cells to migrate toward 10% fetal bovine serum at the bottom of the well. The migrated cells were fixed with 90% ethanol, stained with 0.1% crystal violet, and photographed to count the cells. For the co-culture of A549-M0/M2 and astrocytes, both EGFP-labeled A549-M0/M2 (5×10⁴ cells/well) and mCherry-labeled astrocytes (5×10⁴ cells/well) were cultured in the Transwell inserts for 24 hours. Then, the migrated cells were photographed using a fluorescence microscope to count the cells.

### Example 5. Cell Adhesion Assay

Cells (3×10⁵ cells/well) were seeded in a collagen-coated 24-well plate to allow them to be adhered to the bottom of the plate for 30 minutes. After culture, the plate was washed with PBS twice to remove unadhered cells, and the adhered cells were fixed with 90% ethanol and stained with a 0.1% crystal violet solution. The dye was dissolved in 10% acetic acid, and the percentage of the adhered cells was spectrophotometrically measured at 595 nm.

### Example 6. Spheroid Overlay Analysis

To generate a spheroid, 20% Methocel (Sigma-Aldrich, St. Louis, MO, USA) and 1% Matrigel (BD Biosciences, Franklin Lakes, NJ, USA) were adhered to the lid of a 100 mm petri dish and cultured at 37° C. for 2 days. Then, the spheroid overlaid a feeder cell layer of mCherry-labeled astrocytes. After 12 hours, the invasion of A549 spheroid was visualized using a fluorescence microscope.

### Example 7. Western Blotting

A cell lysate was prepared using a dissolution buffer (50 mM Tris-Cl pH 7.4, 150 mM NaCl, 1 mM EDTA, 1% Triton X-100) containing a protease inhibitor (Sigma-Aldrich). Afterward, a protein (50-100 μg) was isolated by SDS-PAGE, transferred to a PVDF membrane, and incubated with a primary antibody and an HRP-binding secondary antibody (Bio-Rad; Hercules, CA, USA). The visualization of protein bands was performed using the Miracle-Star Western Blot Detection System (iNtRON Biotechnology, Seongnam, Korea). Antibodies such as ZEB1 (1:1000 dilution, #NBP1-05987, Novus Biologicals; Centennial, CO, USA), Vimentin (1:1000 dilution, #sc-5565, Santa Cruz Biotechnology; Dallas, TX, USA), E-cadherin (1:1000 dilution, #sc-8426, Santa Cruz Biotechnology), N-cadherin (1:250 dilution, #sc-7939, Santa Cruz Biotechnology), Twist1 (1:1000 dilution, #90445, Cell Signaling Technology, Danvers, MA, USA), Noggin (1:1000 dilution, #sc-293439, Santa Cruz Biotechnology) and β-actin (1:5000 dilution, #BS6007M, Bioworld Technology; St. Louis Park, MN, USA) were purchased and used.

### Example 8. RNA Sequencing

A total RNA concentration was determined using Quant-IT RiboGreen (Invitrogen). To evaluate the integrity of total RNA, a sample was placed on a TapeStation RNA ScreenTape (Agilent Technology, Santa Clara, CA, USA), and only a high-quality RNA agent having RIN more than 7.0 was used to construct RNA libraries. The libraries were prepared independently for each sample using 1 μg of total RNA and an Illumina TruSeq Stranded mRNA Sample Prep kit (Illumina, San Diego, CA, USA). In the first step, the purification of poly A containing an mRNA molecule was performed using poly A-attached magnetic beads. After purification, the mRNA was fragmented into small pieces using a divalent cation at an elevated temperature.

After replicating the RNA fragment cleaved using SuperScript II reverse transcriptase (Invitrogen) and random primers to the first strand cDNA, the second strand cDNA was synthesized using DNA polymerase I, RNase H, and dUTP. In addition, such cDNA fragments underwent end repairing, single ‘A’ base addition, and adapter ligation. The product was then purified and concentrated by PCR to generate the final cDNA library. The library was quantified using a KAPA Library Quantification kit for Illumina Sequencing platforms (Kapa Biosystems, Wilmington, MA, USA), and verified using a TapeStation D1000 ScreenTape (Agilent). Two-way (2×100 bp) sequencing was performed on a library indexed by Macrogen (Seoul, Korea) using Illumina NovaSeq (Illumina), confirming a gene showing differential expression between A549-M0 and A549-M2.

### Example 9. Real-Time Quantitative Reverse Transcription PCR (qRT-PCR)

Total RNA was isolated from A549-M0 and A549-M2 cells using an AccuPrep Universal RNA Extraction kit (Bioneer). Reverse transcription was performed using an ELPIS RT Prime kit (Elpis-Biotech, Daejeon, Korea). qRT-PCR analysis was performed using a BioFACT A-Star Real-time PCR kit containing SFCgreen I (BioFACT, Daejeon, Korea). An mRNA level was normalized with ribosome protein L32(RPL32) mRNA, and quantitative data was calculated using a 2^{-ΔΔCt} method.

The primer sequence used in the qRT-PCR analysis in the present invention is shown in Table 2 below.

TABLE 2

| | | SEQ ID | | SEQ ID |
|---|---|---|---|---|
| genes | forward (5′→3′) | NO: | reverse (5′→3′) | NO: |
| RPL32 | ACAAAGCACATGCTGCCCAGTG | 8 | TTCCACGATGGCTTTGCGGTTC | 9 |
| CRB3 | CTTCTGCAAATGAGAATAGCACTG | 10 | GACCACGATGATAGCAGTGATGG | 11 |
| CDH1 | GCCTCCTGAAAAGAGAGTGGAAG | 12 | TGGCAGTGTCTCTCCAAATCCG | 13 |
| PARD3 | CGGTCAAAAGAGAACCACGCAG | 14 | CATTCACCCGAAGCCTTCCATC | 15 |
| PATJ | ACAAGGCAGATTTGACGACCTGG | 16 | CTTTGAGCCACAACAGGAAGGTC | 17 |
| SNAI1 | CTGAGGCCAAGGATCTCCAG | 18 | TGCAGTATTTGCAGTTGAAGGC | 19 |
| TWIST2 | GCAAGATCCAGACGCTCAAGCT | 20 | ACACGGAGAAGGCGTAGCTGAG | 21 |
| ZEB1 | GGCATACACCTACTCAACTACGG | 22 | TGGGCGGTGTAGAATCAGAGTC | 23 |
| TWIST1 | GCCAGGTACATCGACTTCCTCT | 24 | TCCATCCTCCAGACCGAGAAGG | 25 |
| FN1 | ACAACACCGAGGTGACTGAGAC | 26 | GGACACAACGATGCTTCCTGAG | 27 |
| VIM | AGGCAAAGCAGGAGTCCACTGA | 28 | ATCTGGCGTTCCAGGGACTCAT | 29 |
| CDH2 | ATCTCGGGTCAGCTGTCGG | 30 | GGCTATCTGCTCGCGATCC | 31 |
| SNAI2 | ATCTGCGGCAAGGCGTTTTCCA | 32 | GAGCCCTCAGATTTGACCTGTC | 33 |
| ZEB2 | AATGCACAGAGTGTGGCAAGGC | 34 | CTGCTGATGTGCGAACTGTAGG | 35 |
| NOG | GCCAGCACTATCTCCACATCCG | 36 | AGCAGCGTCTCGTTCAGATCCT | 37 |

### Example 10. Statistical Analysis

Unless otherwise stated, data was analyzed by the Student's t-test and ANOVA using GraphPad Prism (La Jolla, CA, USA). P value <0.05 was considered statistically significant.

### Experimental Examples

### Experimental Example 1. Confirmation of Brain Colonization Ability of Lung Cancer Cells

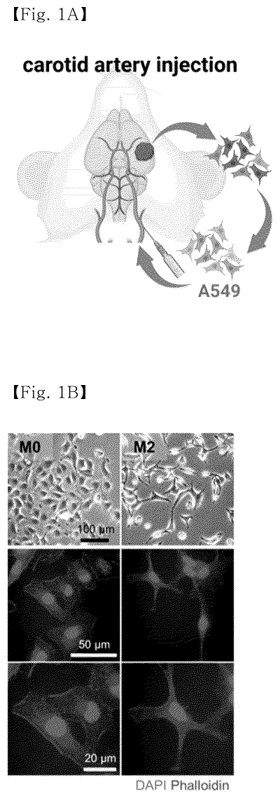

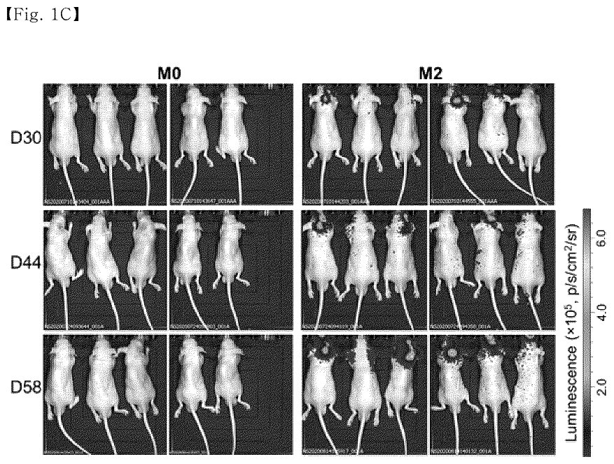

To experimentally reproduce the metastatic brain colonization of lung cancer cells, A549 human lung adenocarcinoma cells were injected into the brain via the carotid artery of athymic nude mice. After 8 weeks, brain tumor formation was confirmed using in vivo bioluminescence imaging, and brain-colonized tumors were isolated and dissociated into single-cell suspensions, which were then retransplanted into the brain of another mouse using the same method. This process is schematically illustrated in FIG. 1A. After two cycles of brain transplantation and isolation, A549-M2 cells were established, and mother A549 cells were named A549-M0. A549-M0 and M2 cells had little difference in cell morphology, and as shown in FIG. 1B, the M2 cells were more pointed and longer than the M0 cells. When these two types of cancer cells were injected again into the brain via the carotid artery, as shown in FIG. 1C, the M2 cells were much better colonized in the brain than the M0 cells. These results suggest that lung cancer cells with high brain colonization potential can be selectively isolated and established through repeated brain transplantation.

### Experimental Example 2. Confirmation of Reduced Growth and Migration of Brain-Colonized Lung Cancer Cells

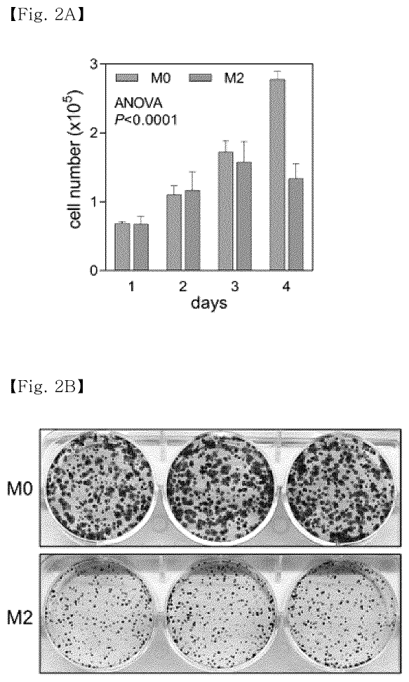

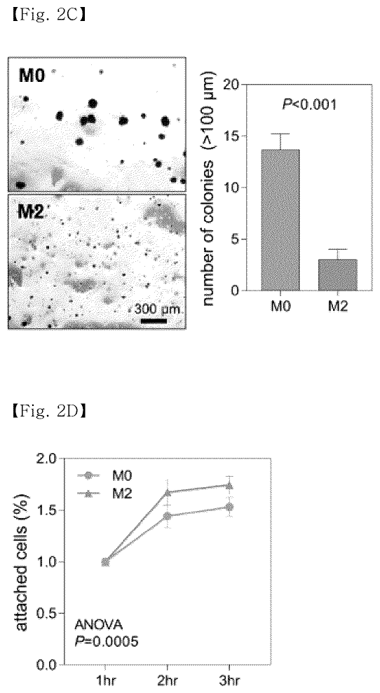

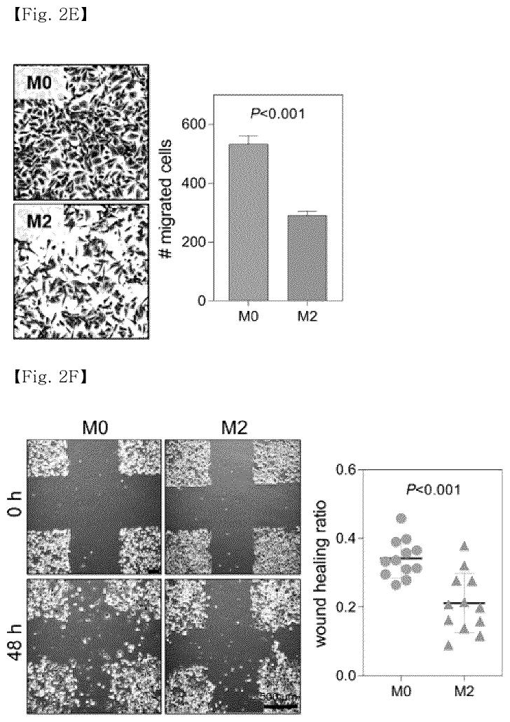

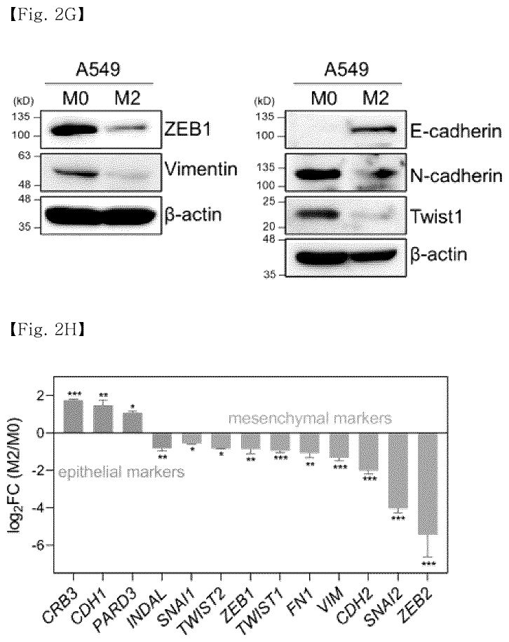

First, the growth rates of the A549-M0 and M2 cells were compared. As a result, as shown in FIG. 2A, the analysis of cell counting showed that M2 cells grew more slowly than MO cells, and as shown in FIG. 2B, M2 cells formed significantly fewer and smaller colonies than MO cells in the clonogenic assay. As shown in FIG. 2C, in the soft agar colony assay, M2 cells could not form as many colonies as M0 cells. From this, it can be seen that the proliferative ability of metastatic lung cancer cells may decrease after brain colonization. The low colony formation of the M2 cells in the soft agar indicates that these cells are highly dependent on anchorage or adhesion to the bottom. This is supported by the results of FIG. 2D, which confirmed that M2 cells adhered better to a collagen-coated plate than M0 cells, in the cell adhesion assay.

In addition, as shown in FIG. 2E, M2 cells exhibited lower motility than M0 cells in the Transwell migration assay, and as shown in FIG. 2F, the same result was shown in the wound healing assay. High adhesion and low motility are key indicators of the mesenchymal-to-epithelial transition (MET). Therefore, the expression levels of mesenchymal and epithelial markers in M0 and M2 cells were confirmed through Western blotting and qRT-PCR. As a result, as shown in FIG. 2G, it was confirmed that the protein expression levels of mesenchymal markers such as ZEB1, Vimentin, N-cadherin, and Twist1 are relatively lower in M2 cells, compared to M0 cells. In contrast, the protein expression of the epithelial marker E-cadherin was higher in M2 cells, compared to M0 cells. The qRT-PCR results were similar to the Western blotting results, and as shown in FIG. 2H, the mRNA level of the mesenchymal marker is lower in M2 cells than M0 cells and the mRNA level of the epithelial marker is higher in M2 cells than M0 cells, which was normalized to the RPL32 mRNA level (*P<0.05, **P<0.01, ***P<0.001). These results mean that metastatic lung cancer cells can lose proliferation and migration activity and gain adhesion activity, and undergo MET after brain colonization.

Experimental Example 3. Confirmation of Low Invasiveness of Brain-Colonized Lung Cancer Cells

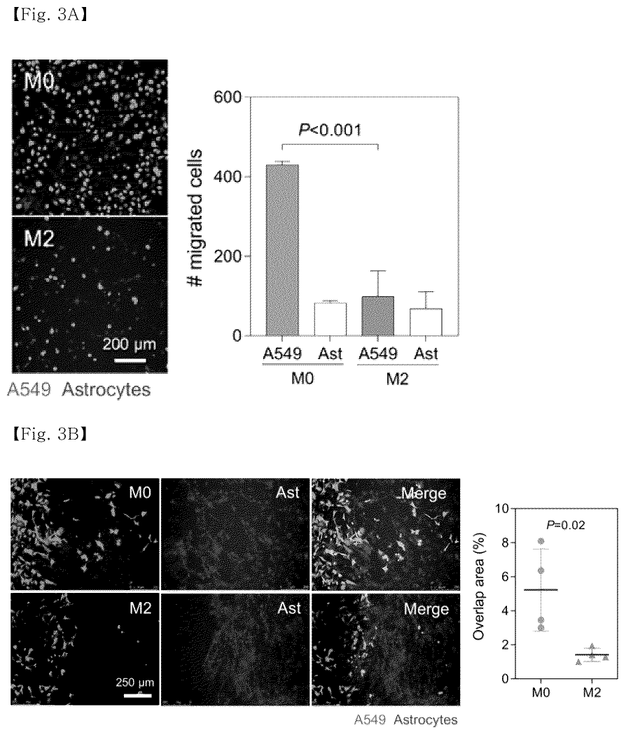

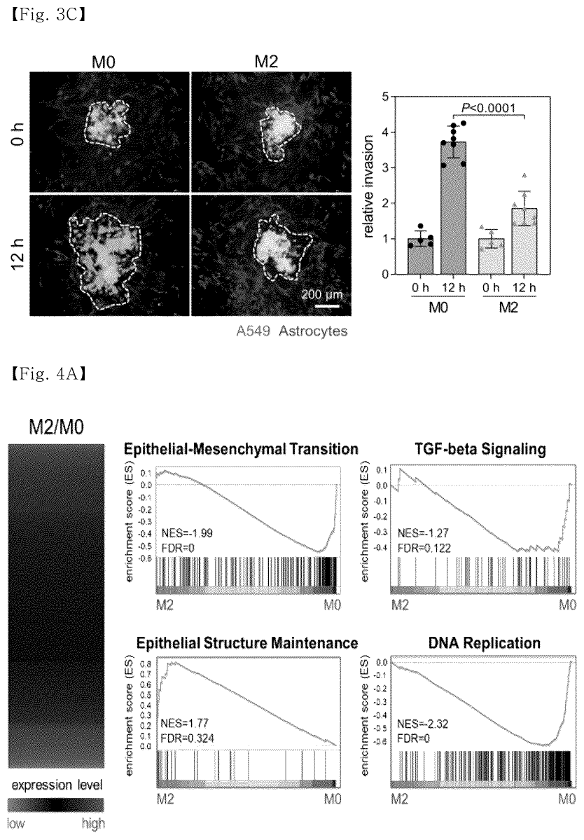

Interactions between lung cancer cells and astrocytes during brain colonization were investigated using a co-culture system. To this end, both GFP-labeled lung cancer cells and mCherry-labeled astrocytes were seeded into upper inserts of a Transwell system, and the migrated cells were observed using a fluorescence microscope. As a result, as shown in FIG. 3A, the motility of the astrocytes was independent of the co-cultured lung cancer cells. However, when co-cultured with astrocytes, M0 cells migrated much better than M2 cells. The lung cancer cells and the astrocytes were then seeded at both sides of a Culture-Insert 2 Well in a 35 mm plate, and were forced to migrate toward the center, leading to wound closing. As a result, as shown in FIG. 3B, M0 cells interacted and were mixed well with astrocytes, whereas M2 cells did not. As shown in FIG. 3C, when spheroids generated from M0 or M2 cells were overlaid on a feeder layer, the M0 spheroids expanded by invading feeder astrocytes better than the M2 spheroids. These results suggest that metastatic lung cancer cells become less invasive after brain colonization. In the co-culture with astrocytes, M0 cells exhibited migratory and invasive properties, whereas M2 cells showed a tendency to adhere to or maintain a contact with astrocytes, promoting their colonization in the brain microenvironment.

### Experimental Example 4. Confirmation of Inhibition of Lung Cancer Cell Migration and Invasion by Reduced Noggin Upon Brain Colonization

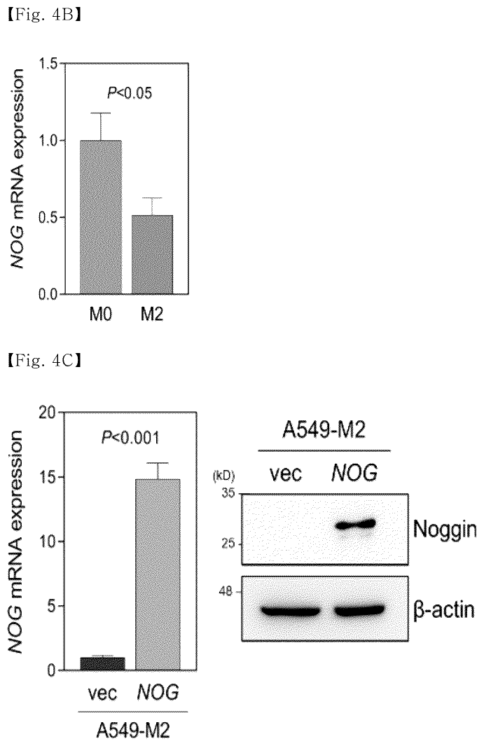

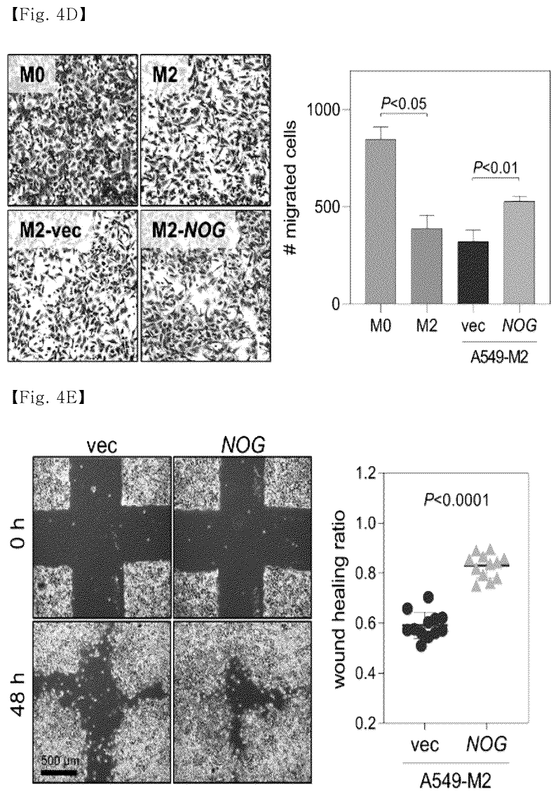

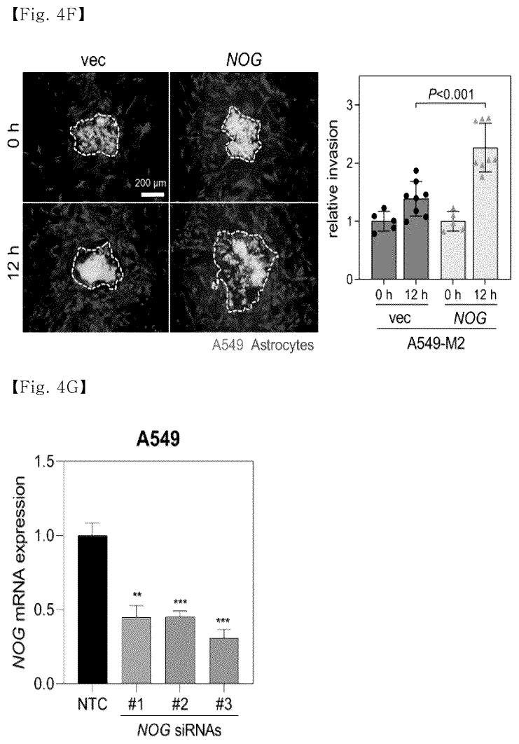

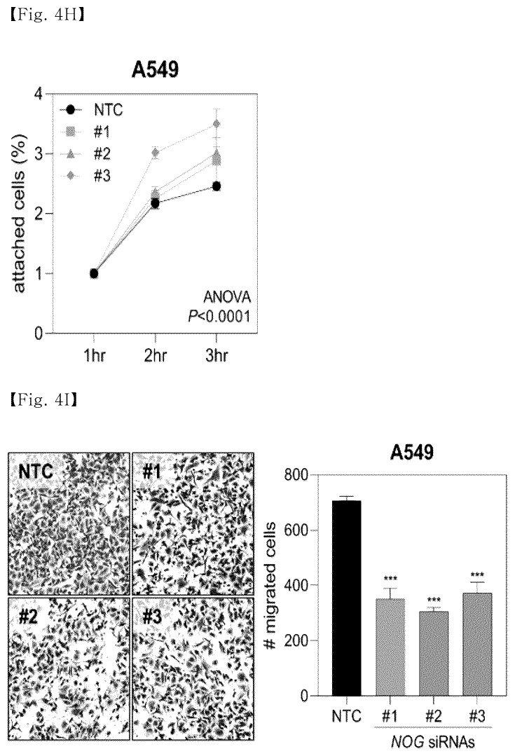

To detect changes in gene expression levels, as shown in FIG. 4A, RNA sequencing was performed using M0 and M2 cells. In the gene set enrichment analysis (GSEA), genetic signatures, including epithelial-mesenchymal transition (normalized enrichment score, NES=−1.99; false discovery rate, FDR=0), TGF-beta signaling (NES=−1.27) and DNA replication (NES=−2.32), were found to be depleted in M2 cells. However, the genetic signature of epithelial structure maintenance (NES=1.77) was enriched in M2 cells, which was consistent with the induction of mesenchymal-to-epithelial transition and growth inhibition of M2 cells as described above. GSEA showed that genetic signatures associated with epithelial-mesenchymal transition (EMT) and TGFβ signaling were downregulated in A549-M2 cells compared to mother cells, which was consistent with data confirmed from cell migration and invasion assays. These results mean that M2 cells undergo MET, which is essential for the later stage of metastatic colonization. Cancer cells undergoing MET have increased cell-cell and cell-matrix adhesions and a reduced ability to migrate and invade surrounding tissue. In the early stage of metastasis, EMT is helpful, but cells undergoing MET have greatly enhanced colonization potential. Considering that the mouse model used in the present invention is optimized for metastatic colonization, M2 cells known to undergo MET-favorable colonization were selected, and as shown in FIG. 4B, among the genes downregulated in M2 cells, Noggin, which is a BMP antagonist, was selected for further study.

The overexpression of Noggin in M2 cells was confirmed as shown in FIG. 4C, and as a result of confirming the effect on cancer cell migration, as shown in FIGS. 4D and 4E, Noggin overexpression restored the inhibited migration of M2 cells in the Transwell (FIG. 4D) and wound healing assays (FIG. 4E). In the spheroid overlay analysis, as shown in FIG. 4F, the Noggin-overexpressed M2 cells expanded by much better invasion toward the feeder astrocytes than the control cells. In addition, Noggin knockdown by siRNA in the control A549 cells was confirmed in FIG. 4G (normalized to the RPL32 mRNA level, **P<0.01, ***P<0.001), and such Noggin knockdown promoted cell adhesion as shown in FIG. 4H, and inhibited cell migration as shown in FIG. 4I. These results demonstrated that a decrease in Noggin during brain colonization may inhibit the migration and invasion of metastatic lung cancer cells.

It should be understood by those of ordinary skill in the art that the above description of the present invention is exemplary, and the exemplary embodiments disclosed herein can be easily modified into other specific forms without departing from the technical spirit or essential features of the present invention. Therefore, it should be interpreted that the exemplary embodiments described above are illustrative in all aspects, and not restrictive.

### Accession Number

Name of Depository Institute: Korean Collection for Type Culture (KCTC)
Accession Number: KCTC 15581BP
Date of Deposition: 20230831

1-8. (canceled)

9. A method of preparing the brain metastatic lung cancer cell line, comprising:
(a) injecting an isolated lung cancer cell line into the carotid artery of an animal model;
(b) isolating brain tissue from the cell line-injected animal model, isolating the lung cancer cell line metastasized to the brain, and culturing the cell line; and
(c) injecting the isolated and cultured brain metastatic lung cancer cell line into the carotid artery of another animal model, and isolating the lung cancer cell line colonized in the brain.

10. The method of claim 9, wherein, in (a), the lung cancer cell line is a human lung cancer cell line A549.

11. A brain metastatic lung cancer animal model into which the brain metastatic lung cancer cell line is transplanted.

12. The brain metastatic lung cancer animal model of claim 11, wherein the animal model has lung cancer cells colonized in the brain.

13-18. (canceled)

19. A method of inhibiting brain metastasis of lung cancer in a subject in need thereof, comprising administering a therapeutically effective amount of a composition comprising an expression or activation inducer of Noggin protein or its mRNA as an active ingredient.

20. The method of claim 19, wherein the expression or activation inducer of Noggin protein or its mRNA is a recombinant vector containing Noggin gene, or Noggin protein.

21. The method of claim 19, wherein the composition inhibits the brain colonization of lung cancer cells.

22. (canceled)

23. The brain metastatic lung cancer animal model of claim 11, wherein the lung cancer cell line is a human lung cancer cell line A549.

24. The brain metastatic lung cancer animal model of claim 11, wherein the brain metastatic lung cancer cell line is A549-BM under Accession No. KCTC 15581BP.

25. The brain metastatic lung cancer animal model of claim 11, wherein the lung cancer cell line is administered into the carotid artery and colonized in the brain.

26. The brain metastatic lung cancer animal model of claim 11, wherein the brain metastatic lung cancer cell line is reduced in motility and invasiveness and increased in adhesion during brain colonization.

27. The brain metastatic lung cancer animal model of claim 11, wherein the brain metastatic lung cancer cell line undergoes mesenchymal-to-epithelial transition (MET).

28. The brain metastatic lung cancer animal model of claim 11, wherein the expression levels of one or more proteins selected from ZEB1, Vimentin, N-cadherin, and Twist1, or their mRNAs are lower than those in a non-metastatic lung cancer cell line.

29. The brain metastatic lung cancer animal model of claim 11, wherein the expression level of E-cadherin protein or its mRNA is higher than that in a non-metastatic lung cancer cell line.

30. The method of claim 19, wherein the method is measuring the expression level of Noggin protein or its mRNA in a biological sample isolated from the subject;
determining that lung cancer has metastasized to the brain, when the expression level of Noggin protein or its mRNA is reduced compared to a control; and
administering the composition into the subject

31. The method of claim 30, wherein the biological sample is brain tissue-derived cells.


SEQUENCE LISTING (37 sequences)

SEQ ID NO: 1 - protein, 232 aa, Homo sapiens
MERCPSLGVTLYALVVVLGLRATPAGGQHYLHIRPAPSDNLPLVDLIEHPDPIFDPKEKDLNETLLRSLLGGHYDPGFMATSPPEDRPGGGGGAAGGAEDLAELDQLLRQRPSGAMPSEIKGLEFSEGLAQGKKQRLSKKLRRKLQMWLWSQTFCPVLYAWNDLGSRFWPRYVKVGSCFSKRSCSVPEGMVCKPSKSVHLTVLRWRCQRRGGQRCGWIPIQYPIISECKCSC

SEQ ID NO: 2 - RNA, 19 nt, synthetic construct
  source 1..19: mol_type = other RNA
agagagacttattctggtt

SEQ ID NO: 3 - RNA, 19 nt, synthetic construct
  source 1..19: mol_type = other RNA
aaccagaataagtctctct

SEQ ID NO: 4 - RNA, 19 nt, synthetic construct
  source 1..19: mol_type = other RNA
cattcttcggaaagtgttt

SEQ ID NO: 5 - RNA, 19 nt, synthetic construct
  source 1..19: mol_type = other RNA
aaacactttccgaagaatg

SEQ ID NO: 6 - RNA, 19 nt, synthetic construct
  source 1..19: mol_type = other RNA
gaagctgcggaggaagtta

SEQ ID NO: 7 - RNA, 19 nt, synthetic construct
  source 1..19: mol_type = other RNA
taacttcctccgcagcttc

SEQ ID NO: 8 - DNA, 22 bp, synthetic construct
  source 1..22: mol_type = other DNA
acaaagcacatgctgcccagtg

SEQ ID NO: 9 - DNA, 22 bp, synthetic construct
  source 1..22: mol_type = other DNA
ttccacgatggctttgcggttc

SEQ ID NO: 10 - DNA, 24 bp, synthetic construct
  source 1..24: mol_type = other DNA
cttctgcaaatgagaatagcactg

SEQ ID NO: 11 - DNA, 23 bp, synthetic construct
  source 1..23: mol_type = other DNA
gaccacgatgatagcagtgatgg

SEQ ID NO: 12 - DNA, 23 bp, synthetic construct
  source 1..23: mol_type = other DNA
gcctcctgaaaagagagtggaag

SEQ ID NO: 13 - DNA, 22 bp, synthetic construct
  source 1..22: mol_type = other DNA
tggcagtgtctctccaaatccg

SEQ ID NO: 14 - DNA, 22 bp, synthetic construct
  source 1..22: mol_type = other DNA
cggtcaaaagagaaccacgcag

SEQ ID NO: 15 - DNA, 22 bp, synthetic construct
  source 1..22: mol_type = other DNA
cattcacccgaagccttccatc

SEQ ID NO: 16 - DNA, 23 bp, synthetic construct
  source 1..23: mol_type = other DNA
acaaggcagatttgacgacctgg

SEQ ID NO: 17 - DNA, 23 bp, synthetic construct
  source 1..23: mol_type = other DNA
ctttgagccacaacaggaaggtc

SEQ ID NO: 18 - DNA, 20 bp, synthetic construct
  source 1..20: mol_type = other DNA
ctgaggccaaggatctccag

SEQ ID NO: 19 - DNA, 22 bp, synthetic construct
  source 1..22: mol_type = other DNA
tgcagtatttgcagttgaaggc

SEQ ID NO: 20 - DNA, 22 bp, synthetic construct
  source 1..22: mol_type = other DNA
gcaagatccagacgctcaagct

SEQ ID NO: 21 - DNA, 22 bp, synthetic construct
  source 1..22: mol_type = other DNA
acacggagaaggcgtagctgag

SEQ ID NO: 22 - DNA, 23 bp, synthetic construct
  source 1..23: mol_type = other DNA
ggcatacacctactcaactacgg

SEQ ID NO: 23 - DNA, 22 bp, synthetic construct
  source 1..22: mol_type = other DNA
tgggcggtgtagaatcagagtc

SEQ ID NO: 24 - DNA, 22 bp, synthetic construct
  source 1..22: mol_type = other DNA
gccaggtacatcgacttcctct

SEQ ID NO: 25 - DNA, 22 bp, synthetic construct
  source 1..22: mol_type = other DNA
tccatcctccagaccgagaagg

SEQ ID NO: 26 - DNA, 22 bp, synthetic construct
  source 1..22: mol_type = other DNA
acaacaccgaggtgactgagac

SEQ ID NO: 27 - DNA, 22 bp, synthetic construct
  source 1..22: mol_type = other DNA
ggacacaacgatgcttcctgag

SEQ ID NO: 28 - DNA, 22 bp, synthetic construct
  source 1..22: mol_type = other DNA
aggcaaagcaggagtccactga

SEQ ID NO: 29 - DNA, 22 bp, synthetic construct
  source 1..22: mol_type = other DNA
atctggcgttccagggactcat

SEQ ID NO: 30 - DNA, 19 bp, synthetic construct
  source 1..19: mol_type = other DNA
atctcgggtcagctgtcgg

SEQ ID NO: 31 - DNA, 19 bp, synthetic construct
  source 1..19: mol_type = other DNA
ggctatctgctcgcgatcc

SEQ ID NO: 32 - DNA, 22 bp, synthetic construct
  source 1..22: mol_type = other DNA
atctgcggcaaggcgttttcca

SEQ ID NO: 33 - DNA, 22 bp, synthetic construct
  source 1..22: mol_type = other DNA
gagccctcagatttgacctgtc

SEQ ID NO: 34 - DNA, 22 bp, synthetic construct
  source 1..22: mol_type = other DNA
aatgcacagagtgtggcaaggc

SEQ ID NO: 35 - DNA, 22 bp, synthetic construct
  source 1..22: mol_type = other DNA
ctgctgatgtgcgaactgtagg

SEQ ID NO: 36 - DNA, 22 bp, synthetic construct
  source 1..22: mol_type = other DNA
gccagcactatctccacatccg

SEQ ID NO: 37 - DNA, 22 bp, synthetic construct
  source 1..22: mol_type = other DNA
agcagcgtctcgttcagatcct